(19) United States
(12) Patent Application Publication
(10) Pub. No.: US 2004/0106695 A1
(43) Pub. Date: Jun. 3, 2004

(54) Method of tooth whitening with synthetic resin in white liquid nail-polish type

(76) Inventor: Despina Kostomiri, Pefki Attikis (GR)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/467,597

(22) PCT Filed: Feb. 6, 2002
(86) PCT No.: PCT/GR02/00006

(30) Foreign Application Priority Data
Feb. 7, 2001 (GR) 20010100059

(51) Int. Cl. A61F 2/00

(52) U.S. Cl. 523/115

(57) ABSTRACT

The method of tooth whitening using white, liquid, nail-polish-type synthetic resins, is characterized by the following: We apply-paint, in any manner, like nail polish—the external tooth surface, with a white, liquid resin in many shades that is not currently commercially available. The tooth surface can be already elaborated with known methods.

The invention refers to a method and product manufacturing for teeth whitening, based on the utilization of white, nail-polish-type, liquid synthetic resin produced in many shades.

Tooth whitening is a known technique that is based until today on the utilization of whitening gels or laser.

Both existing methods have the following disadvantages:

They do not achieve, in all cases, the desired (preselected) whiteness of the teeth, because they remove only the acquired stains and can restore the original color in only a small percentage of people (the genetically determined color of the enamel, which usually is not white).

If there are problems with tooth fractures or old, unsightly fillings or problems with recession of the gingiva (cervical recessions) they cause sensitivities of the tooth and their application must stop immediately without the option of using them in the future.

As far as lasers and their application, special protection of the gingiva is required so as not to create health problems (in the gingiva).

The same apply to the whitening gels, but in a lesser degree.

Another disadvantage for gels is the fact that to achieve a result (if any) they have to be applied daily for several days.

It is noted that, it is not allowed to apply repeatedly and frequently whitening gels in order to preserve the shade of white, because the strength of the enamel is reduced.

The results, achieved with the gels, are not long term.

In a short time, usually a few months, the teeth return to their original color condition.

If there are white fillings of resins on the tooth, they can change only the color of the enamel and not of the filling. So after the whitening there is a difference in color between filling and enamel (unaesthetic result).

In conclusion, the techniques of using gels and lasers cannot produce a desirable (preselected) whiteness, but in the best case, the original (inherited) color of the enamel (which usually is not white).

To achieve this result, they must be applied more than once (mainly gels) and it is possible to create problems with gingiva and sensitivity to the teeth, and their results are limited in time.

In this invention, whitening is achieved by using a liquid, nail-polish-type, white synthetic resin with several shades of white, which is not commercially available in this form, in order to be used with this method.

As well as, there is no other dental material in liquid form, that can be used with this method.

And this is because the idea of using this method of tooth whitening did not exist up to now.

The advantages of tooth whitening with synthetic resin (or other dental material in liquid form) are the following:

The tone of white we preselected is achieved with only one application. It is independent on the inherited color of the enamel.

We do not have problems with sensitivity, because resins function in dental practice for therapeutic reasons (white fillings) and are recommended materials for therapeutic reasons.

We do not have therapeutic problems with the gingiva, because they are materials, compatible with the oral cavity (are used for therapeutic reasons in the mouth).

We have a many years, both clinical and laboratory, experience with resins, because they are used for many years in therapeutic restorations of dental fillings, having long term results.

If there are white fillings, in the teeth, they will change the color of the filing and not only of the tooth, given the fact that they are materials, similar to that of the filling.

Using this invention, the results of tooth whitening are long term.

The invention is described below, presenting the methodology:

Short Description

Stage 1: Teeth-treatment by using known methods.

Stage 2: Painting in a nail-polish-type of a liquid white resin (in many shades). New method, new product, new application.

Stage 3: Photopolymerization of the liquid white resin of many shades.

### DETAILED DESCRIPTION

Stage 1 Teeth-Treatment, by Using Known Methods:

Indicatively, one of the most known methods of teeth-treatment, which is used for therapeutic reasons, while in this invention is for whitening, is described below.

(The teeth are cleaned. We pass a transparent isolating strip to the tooth in order to check the exact limits of whitening. The enamel is etched with H3PO4 (orthophosphoric acid). We rinse with water. We dry. We apply a one-phase adhesive factor and photopolymerize). It is noted that the above method of tooth preparation is not limited in accepting the invention of teeth whitening through painting in a nail-polish-type, which is described in Stage 2.

Stage 2 Painting in a Nail-Polish-Type of the Liquid White Resin, Resulting Whitening.

We apply (paint in any manner) in a nail-polish-type on the external surface of the tooth (which has been treated or properly prepared) liquid white resin.

In this invention, which is the whitening of teeth through the painting with liquid resin, it must be noted that, there are no commercially available nail-polish-type, white, liquid resins, nor any similar white liquid products because their only existing reason is tooth whitening.

Stage 3 Photopolymerization of the Liquid White Resin of Stage 2

We photopolymerize the liquid white resin with which we have painted the external surface of the tooth in Stage 2, without considering photopolymerization as a limiting factor of stabilizing the liquid white resin on the external surface of the tooth.

Stages 1 and 3 are not limiting for application of Stage 2.

The synthetic resin can be substituted by any material of similar form and use.

Such a material can result by the total substitution of the synthetic resin or from the combination of the synthetic resin with other materials, for example, by the combination of the synthetic resin with a glass ionomer material in a liquid, white, nail-polish-type final form.

It is noted that the proposed new method can lead to the production of dental materials with the following characteristics:

A. To be applied on teeth without the need of any prior preparation

B. To be applied on teeth without the need of photopolymerization

C. To be applied on teeth without the need of preparation and photopolymerization.

Also the replacement of the synthetic resin with another similar, in the form and utilization, material, could lead to the production and disposal of a cosmetic material, without being necessarily a dental one and used by a dentist.

1) The method of tooth whitening, using liquid, white, nail-polish-type synthetic resins, characterized by: Stage 1. We elaborate the external tooth surface with known methods such as: We clean the teeth. We pass an isolating strip. We etch the enamel. We apply an adhesive factor. Stage 2. We apply-paint, in any manner, in a nail-polish-type to the external tooth surface with a white, liquid resin of many shades, that is not currently commercially available. Stage 3. We photopolymerize the white liquid resin.

2) According to claim 1 the teeth whitening is made with a white, liquid, nail-polish-type resin, a method that guarantees a 100% whiteness or the desired whiteness as well as long term results.

3) This product, the liquid, white, nail-polish-type resin, as described in claims 1 and 2 is used for the first time for the teeth whitening and has been commercially available up to now for this methodology.

4) Use of the liquid, white, nail-polish-type resin as described in claims 1 and 3 for the teeth whitening.

5) The tooth whitening method, as described in claim 1 characterized by the fact that the white, liquid, nail-polish-type synthetic resin can be applied on the external surface of the tooth-like a nail polish, without using Stage 1 of the method described in claim 1 of the preliminary preparation of the tooth surface.

6) The tooth whitening method as described in claim 1, is characterized by the fact that the white, liquid resin is applied on the external tooth surface, in a nail-polish-type, without being followed by the Stage 3 photopolymerization.

7) The tooth whitening method, as described in claim 1, is characterized by the fact that the white, liquid resin is applied on the external tooth surface in a nail-polish-type directly without the tooth preparation of Stage 1 or the photopolymerization of Stage 3.

8) The white, liquid resin as described in claims 1 to 7 can be substituted by another material of similar form and use.

9) The white, liquid resin as described in claims 1 to 8 can be combined with a glass ionomer material, to create a nail-polish-type material, of similar form and use.

10) The white, liquid resin as described in claims 1 to 8 can be combined with other material or materials, to create a nail-polish-type material of similar form and use.

11) The white, liquid synthetic resin, as described in claims 1 to 8 can be replaced by another similar material, of the same form and utilization and lead also to the production and disposal of a cosmetic product, without being a dental one and used by dentist.
